(19) United States
(12) Patent Application Publication
(10) Pub. No.: US 2021/0338211 A1
(43) Pub. Date: Nov. 4, 2021

(54) OCULAR INSERTS WITH ANALYTE CAPTURE AND RELEASE AGENTS

(71) Applicant: TearDX LLC, Bayside, NY (US)

(72) Inventors: Eugene Orloff, Bayside, NY (US); Paul V. Braun, Champaign, IL (US); Eric Scott Epstein, Pittsburgh, PA (US)

(21) Appl. No.: 17/306,322

(22) Filed: May 3, 2021

Related U.S. Application Data
(60) Provisional application No. 63/019,008, filed on May 1, 2020.

(51) Int. Cl. A61B 10/00; C12Q 1/6834

(52) U.S. Cl. CPC A61B 10/0045; A61B 2010/0067; C12Q 1/6834

(57) ABSTRACT

An ocular device for insertion into the eye (for example, into a lacrimal punctum or a conjunctival sac) are described herein. The ocular device may continuously capture specific analytes from tear fluid. Several embodiments of the ocular device may have one or more open channels and pores that enable tears to drain naturally through while capturing specific analytes. A mechanism of detecting analytes in bodily fluids using materials comprised of DNAzymes and/or aptamers are described herein. Aptamers are oligonucleotide or peptide molecules that bind to a specific target molecule. DNAzymes are designed to catalyze a number of biological reactions (i.e., RNA cleavage, DNA cleavage, ligation, or phosphorylation reactions). A number of ways an ocular insert may capture analytes in tear fluid in vivo for subsequent in vitro analysis are described herein. Additionally, a specific mechanism for colorimetric detection of analytes using aptamer- and/or DNAzyme-crosslinked hydrogels are described herein.

This application claims priority from U.S. Provisional Application No. 63/019,008 filed May 1, 2020, which is incorporated by reference herein.

### FIELD OF INVENTION

This application is in the field of medical devices.

### BACKGROUND

In recent years, mechanisms have been developed that analyze tears for biomarkers of a variety of diseases through collection of basal tears followed by bioassays (for example, U.S. Patent Application Publication No. 2016/0003786 entitled “Methods of Detecting Cancer”). However, a fundamental issue preventing the effective use of tear fluid for diagnostics is the difficulty of collecting and analyzing tears from patients. The most commonly employed methods, Schirmer strips and microcapillary tubes have major flaws that prevent them from being broadly utilized in clinics. In particular, there are major issues with extraction of proteins from Schirmer strips, whereas microcapillary tubes require long collection times during which patients keep their eyes open while the clinician or technician must steadily hold the tube near the patient's eye. Both methods require relatively long chair times in order to extract relatively small volumes of tears (typically 1-10 μL), making detection of low concentration biomarkers exceedingly difficult. Furthermore, different methods of collection have been shown to yield different collected tear compositions, which, in conjunction with differences in chosen methods of tear biomarker analysis, have produced widely differing, often conflicting, reports of tear composition. Furthermore, these tests only show a snapshot of a patient's medical condition at the time at which the tears were collected, and therefore analysis results may be subject to potentially large fluctuations in tear composition throughout a given day. In order for tear biomarkers to become clinically useful, there is a critical need to establish a less invasive, convenient, standardized, repeatable method for their collection, as well as one that can yield information of a patient's medical condition over a continuous, extended period of time.

Recently, methods have been proposed and claimed for analyzing analytes in tear fluid by collecting tear fluid in a contact lens (for example, U.S. Pat. No. 7,429,465 B2 entitled “Process for Analyzing Tear Fluid” and U.S. Pat. No. 9,320,460 B2 entitled “In-Situ Tear Sample Collection and Testing Using a Contact Lens”) or by continuously monitoring analytes through an embedded sensor in a contact lens (for example, U.S. Pat. No. 6,312,393 B1 entitled “Contact Device for Placement in Direct Apposition to the Conjunctive of the Eye”). A contact lens, however, is not an ideal platform for detecting or capturing analytes in tear fluid, as not everyone can tolerate putting in and removing contact lenses. They may cause irritation to the eye, and they generally cannot be worn continuously for periods longer than a day. In particular, continuous wearing of contact lenses may lead to corneal hypoxia and eye infections. Furthermore, the efficiency of tear collection of contact lenses is imperfect because it is unlikely that all tear fluid impregnates a contact lens before draining through the lacrimal puncta. Another major issue is contact lenses alter the physiology of tears, and therefore they cannot act as a perfectly passive platform for evaluating the physiology of tears. Additionally, the form factor and types of materials used for analyte capture are limited in a contact lens, as any opaque material (for example, iron oxide or gold nanoparticles) would have to be limited to the periphery of the lens so as not to create an optical or aesthetic defect in the lens.

### SUMMARY

An ocular device designed to be inserted into the eye of a patient and continuously capture specific analytes from tear fluid may be described herein. The ocular device may be inserted into a lacrimal punctum, a conjunctival sac, or the like. A number of forms of the device may be described herein, including one that contains one or more open channels and/or pores that enable tears to drain naturally through the device while specific analytes are captured. A specific mechanism of detecting analytes in bodily fluids using materials comprised of DNAzymes and/or aptamers may be described herein. Aptamers may be oligonucleotide or peptide molecules that bind to a specific target molecule. DNAzymes are a class of catalytic oligonucleotides designed to catalyze a number of biological reactions such as RNA cleavage, DNA cleavage, ligation or phosphorylation reactions. A number of ways an ocular insert may capture analytes in tear fluid in vivo for subsequent in vitro analysis may be described herein. Additionally, a specific mechanism for colorimetric detection of analytes using aptamer- and/or DNAzyme-crosslinked hydrogels may be described herein.

### BRIEF DESCRIPTION OF THE DRAWINGS

FIG. 1 is an example of a punctal insert with a hollow channel;

FIG. 2 is an example of a punctal insert used for analyte capture with an open-faced channel;

FIG. 3 is another example of a punctal insert used for analyte capture with a removable porous membrane;

FIG. 4 is another example of a punctal insert used for analyte capture with a removable and irremovable porous membrane;

FIG. 5 is another example of a punctal insert used for analyte capture with open cell microporous foam;

FIG. 6 is an example of a conjunctival sac insert used for analyte capture;

FIG. 7 is an example of a contact lens used for analyte capture;

FIG. 8 is an example method of analyte capture using a lacrimal punctum insert.

FIG. 9 is an example illustration of what this may look like using a fluorescence-based immunoassay; and

FIG. 10 is an example illustration of releasing analytes from a capture agent.

### DETAILED DESCRIPTION OF THE DRAWINGS

An ocular device described herein may be inserted into a puncta (for example, tear ducts), a conjunctival sac, built into a contact lens, or the like. For ocular devices that may be inserted into the puncta at the upper and/or lower eyelids to prevent drainage of basal tears, helping to treat for dry eye syndrome may be described herein. These plugs are typically silicone-based, cause minimal-to-no irritation to the patient, and may be safely left in the puncta for years if not physically dislodged, providing a positive role in the treatment of patients suffering from dry eyes, with little to no negative side effects.

A person's tears may be used to monitor various physiological and/or biochemical states (for example, presence of vitamins, glucose level, and the like), and even to diagnose diseases based on distinct biomarkers present in the tear fluid. Similar to blood, tears are representative of the biochemical composition of the body. There are various kinds of tears including basal tears, reflex tears (for example, resulting from exposure to tear gas), and psychic tears (for example, released when crying). Basal tears are continually produced, and their components may include, but are not limited to, glucose, minerals (for example, iron), vitamins, neurotransmitters, metabolites, amino acids, urea, anti-oxidants, polynucleotides, and many proteins and/or their associated metabolites. Recently, the analysis of toxic heavy metals in tears (for example, lead and arsenic) was shown to be a promising diagnostic tool, as significantly different ranges of concentrations of these heavy metals were detected in representative rural versus urban populations.

An ocular insert with surface-bound capture agents (for example, as antibodies or aptamers) that capture specific analytes in tear fluid for subsequent in vitro analysis after removing the insert, may be advantageous to previously described ocular inserts that have built-in sensors that attempt to perform analysis in vivo. For example, they do not require the complex circuitry, reagents or calibration methods to be confined to the ocular insert and may instead rely on optimizing the actual analysis of captured tear analytes using a much broader range of available techniques and instruments. In addition an ocular insert with surface-bound capture agents that bind to specific tear analytes may be advantageous over ocular inserts that merely collect tears. For example, such methods may only be able to extract relatively small volumes of tears and making analysis of many of the most promising biomarkers of diseases in tears, which have extremely low concentrations (e.g. 1-1000 pg/ml) is exceedingly difficult.

A capture agent may be designed to specifically bind to one (or sometimes several) specific analytes. An example of a capture agent used in molecular biology is an antibody, which specifically binds to one protein out of often thousands typically present in a bodily fluid.

One advantage of surface-bound capture agents, described herein, are the ability to continuously concentrate targeted analytes. This is especially crucial for biomarkers that are present in tears at very low concentrations and thus very difficult to analyze. For example, many cytokines, which are promising diagnostic biomarkers, are present in tears at concentrations at the 1-10 pg/ml scale. For a typical 1-10 microliter tear sample collected by conventional methods, this means there are only 0.001-0.1 pg of cytokine to analyze. On the other hand, an ocular insert that continuously collects low concentration biomarkers such as cytokines, may collect 1-10 pg over the course of the day, making subsequent analysis of the targeted biomarker significantly more reliable.

Punctal inserts, in particular, may offer benefits as a platform for an embedded analyte or biomarker collector because they are low cost, easy to insert, less invasive and less cumbersome to wear and maintain than contact lenses, and, because virtually all tear fluid drains through the two approximately 500 μm wide lacrimal puncta in each eye, thereby having direct interaction with tear fluid without actually coming in contact with the eye. A variety of materials and methods may be used for analyte capture, including opaque materials, because ocular inserts may be designed to not obstruct the vision of the wearer and, with the exception of a contact lens inserts, are not as easily seen by an external observer.

A specific mechanism of detecting analytes in bodily fluids using materials comprised of DNAzymes and/or aptamers may be described herein. DNAzymes are a class of catalytic nucleic acids designed to catalyze a number of biological reactions such as RNA cleavage, DNA cleavage, ligation or phosphorylation reactions. As described herein, “DNAzyme” is meant to broadly encompass all types of systems containing DNAzymes. DNAzymes have been used as a mechanism for designing a variety of different types of sensors with both high sensitivity and high specificity. Their stability in non-controlled environments makes them particularly attractive platforms for sensors.

A number of ways an ocular insert may capture analytes in tear fluid in vivo for subsequent in vitro analysis may be described herein. In another embodiment, an indirect mechanism for detecting and/or quantifying analytes in biofluids by immersing a material comprised of DNAzymes in a biofluid (which may be tears, blood, urine, sweat, or saliva) may be described herein. However, instead of directly relying on analyte capture, the DNAzymes themselves may be analyzed.

Additionally, a specific mechanism for colorimetric detection of analytes using aptamer- and/or DNAzyme-crosslinked hydrogels may be described herein. Aptamers are similar to DNAzymes in that they are oligonucleotides that selectively bind to an analyte, but unlike DNAzymes, they do not catalyze a subsequent reaction. Both aptamer and DNAzyme crosslinked hydrogels have been demonstrated to undergo cleavage upon reacting with a target analyte, leading to dissociation of the hydrogel network. Aptamer and DNAzyme hydrogels have been used as colorimetric biosensors, however, the reported methods are not ideal for operation in vivo. For example, many of these reported methods rely on release of a cargo encapsulated by the DNA-crosslinked hydrogel. However, release of cargo is not ideal for colorimetric reporting in a dynamic and open biological environment (such as in the eye). Another clever device for colorimetric detection of analytes may be comprised of capillary tubes plugged with DNAzyme-crosslinked hydrogels. In the absence of the target analyte, the hydrogel may prevent flow of liquid into the capillary, but when the target analyte is present the DNAzymes may cleave, causing the hydrogel to dissolve and fluid to flow into the capillary, generating a visual change in the capillary that may be detected by the naked eye. This mechanism, however, may not enable easy visual detection in a sensor as compact as one built into an ocular insert.

A mechanism that solves these problems by using a DNAzyme hydrogel whose sole function is to visually conceal a visually distinctive material underneath may be described herein. Upon dissolution or washing away of the DNA-crosslinked hydrogel, the visually distinctive material may be revealed and thus observable by either a detector or the naked eye. For example, such a device may be comprised of fluorescent quantum dots embedded in a polymer (so as to remain mechanically stable), and above this fluorescent material, a DNAzyme-crosslinked hydrogel that may contain a dye that masks the fluorescent quantum dots from an observer and/or from the fluorescent excitation wavelengths of light. Upon dissolution of the opaque hydrogel, the fluorescent quantum dots may be revealed and thus optically detectable. This mechanism may provide a number of benefits over existing methods of DNAzyme and/or aptamer-based colorimetric sensing. For example, it may not rely on releasing any cargo, or on binding fluorescent dyes and quenchers to a DNAzyme (which can be expensive), and instead, may enable the device to be comprised of commercially available color-signaling materials (for example, commercial quantum dots) that have already been optimized for stability and reliability in biological environments.

One application for the methods described herein may be to provide a simple, inexpensive platform for continuously monitoring exposure to pathogens from the environment using easily removable porous lacrimal punctal inserts functionalized for viral capture. These punctal insertions may capture virus particles, bacteria or other pathogens that come in contact with the eye or that may be present in tears due to an existing infection. On a regular basis, the inserts may be removed for analysis and then replaced.

In an exemplary embodiment, a lacrimal punctal insert may be functionalized with a surface-bound capture agent designed to capture one or multiple target analyte(s) from tears. The analyte(s) may be specific organic compounds, pharmacological agents, metal ions, viruses, bacteria, fungi, enzymes, extracellular vesicles, allergens, RNA, DNA, proteins, peptides or other biomarkers.

The punctal insert may or may not be permeable. For example, a permeable punctal insert may be permeable to tears to enable a higher surface area for tears to interact with analyte capture agents. In an alternative example, the punctal insert may only have the capture agent immobilized on a top surface of a completely impermeable punctal plug. The punctal insert may be a specific kind of punctal insert that blocks tears from draining.

The punctal insert may contain a porous and/or permeable material for tear collection and analyte capture, but may not enable tears to continue flowing through the lacrimal puncta. This may, for example, help treat certain forms of dry eyes.

The punctal insert may be entirely comprised of porous and/or permeable materials or that contains one or more channels to enable tears to diffuse or flow through the insert into the lacrimal canaliculi.

The punctal insert may be comprised of one or more of the following scaffold materials that immobilize the capture agents : hydrogels, cellulose fibers, microparticles, porous filter membranes, nano- or microgels (for example, Nanotrap® particles), porous inorganic materials (for example, silicon, silica, ceramics, activated carbon, or graphite), magnetic particles (for example, Mag4C viral capture nanoparticles or Magnetofection™ nanoparticles), non-magnetic particles (for example, carbon, gold, silver, silica or alumina) or molecularly imprinted polymers (used to bind specific analytes based on a lock and key inspired model). The one or more of these materials for analyte capture may contain one or more functional groups. The functional groups may be comprised of one or more of the following: a charged organic molecule or chemical moeity, a charged polymer or oligomer, a hydrophobic polymer or oligomer, an aptamer, an antibody, a peptide, a protein, a bacteriophage, a DNAzyme, a nanozyme, a ligand or a chelator.

The punctal insert may be designed for size exclusion. A size exclusion material may include a porous polymer or inorganic material having a specific pore size, a hydrogel having a specific mesh size based on its swollen state in tear fluid, a size exclusion “gel” comprised of packed hydrophobic particles, or a hydrophobic polymer or inorganic material containing channels of a specific size and geometry (such as the herringbone channels).

The punctal insert may be designed for ion exclusion. For example, ion inclusion may include ion selective membranes.

The punctal insert may be combined with a colorimetric indication mechanism (for example, fluorescence, luminescence, optical absorbance, and/or reflectance) as described by US20180289326A1, which is incorporated herein by reference.

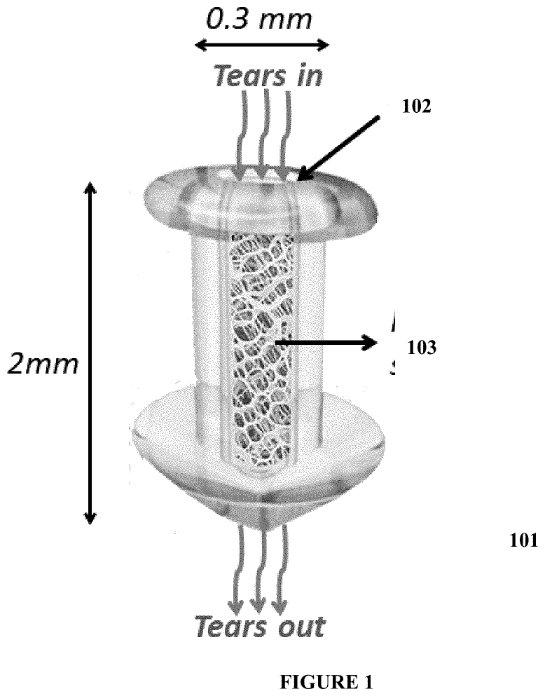

FIG. 1 is an example of a punctal insert with a hollow channel. The punctal insert 101 may include a hollow channel 102 that enables tears to drain naturally through the device. Embedded within the punctal insert 101 is a microporous scaffold material 103 (for example, a porous cellulose membrane), which may contain one or more surface-immobilized capture agents. The immobilized capture agents may capture one or more specifically targeted analytes that flow through the punctal insert 101 through naturally draining tears. The punctal insert 101 may be approximately 2 mm in length and 0.3 mm in width.

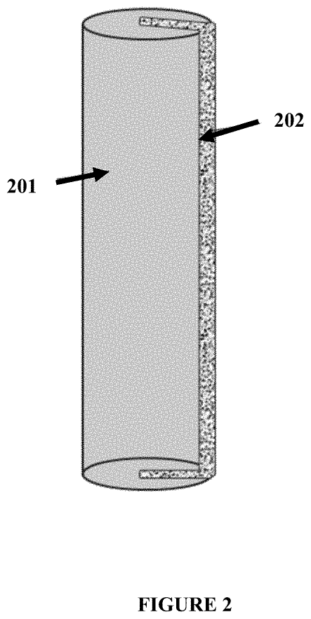

FIG. 2 is an example of a lacrimal punctal insert used for analyte capture with an open-faced channel. The lacrimal punctal insert 201 may have an open-faced channel 202. The open-faced channel 202 may contain a capture-agent-immobilizing scaffold. The open-faced channel 202 may run through the lacrimal punctal insert 201. A cross-section may look a bit like a “u” with the scaffold in the interior of the “u”.

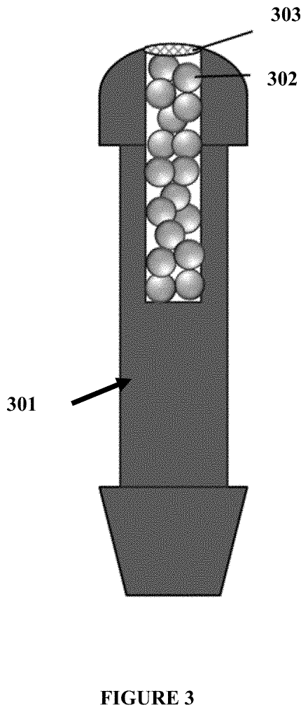

FIG. 3 is another example of a lacrimal punctal insert used for analyte capture that contains a removable porous membrane and prevents tears from flowing into the lacrimal canaliculi. The lacrimal punctal insert 301 may have a removable porous membrane 302 and microparticles 303. The microparticles 303 may contain a surface-bound capture agent. The channel that contains the capture-agent-immobilized microparticles does not extend through the entire plug so that tears do not drain naturally. The outer portion of the lacrimal punctal insert may be a silicone rubber or some other similar material.

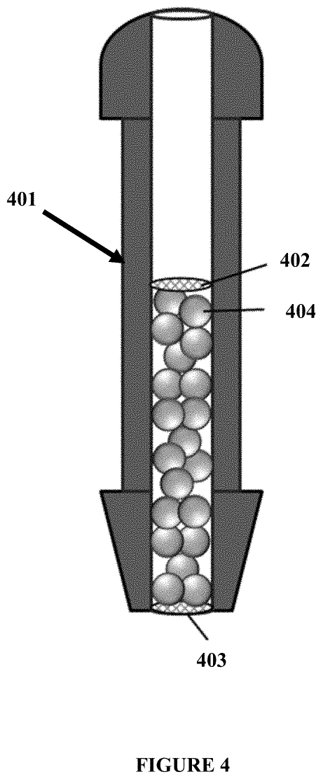

FIG. 4 is another example of a lacrimal punctal insert used for analyte capture with at least one removable porous membrane. The lacrimal punctal insert 401 may have a removable porous membrane 402 and a membrane that may or may not be removable 403. The lacrimal punctal insert 401 may also include microparticles 404 that contain a surface-bound capture agent. The two porous membranes may contain pores that are large enough to enable tears to either diffuse or drain naturally through the device, but that are smaller than the size of the microparticles. Therefore, the microparticles may remain contained inside of the open channel of the lacrimal punctal insert. The removable porous membrane may either be deliberately punctured or removed to release the microparticles for analysis.

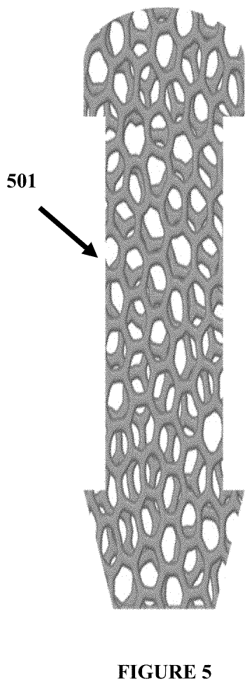

FIG. 5 is another example of a lacrimal punctal insert used for analyte capture with open cell microporous foam. The lacrimal punctal insert 501 may be made entirely of an open cell microporous foam.

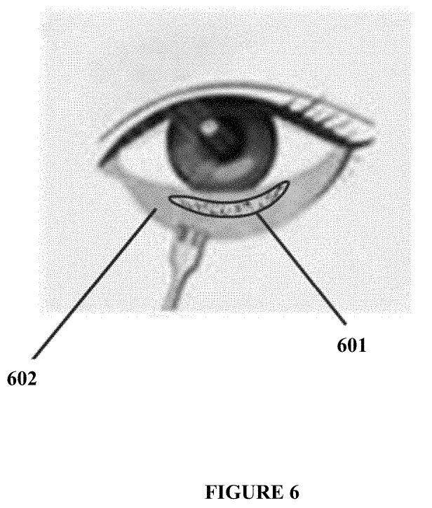

FIG. 6 is an example of a conjunctival sac insert used for analyte capture. The conjunctival sac insert 601 may be inserted into the conjunctival sac 602 of the eye. The conjunctival sac insert 601 may have a permeable insert with a surface-immobilized capture agent.

The punctal insert may include a surface-bound capture agent that specifically binds at least one target analyte from tear fluid. There may be several options for surface-bound capture agents. For example, capture agents may bind directly to the surface of the punctal insert, either the outer or inner punctal insert may contain a channel or pore. In another example, capture agents may bind to a separate scaffold material that is contained with an open channel within the punctal insert. An example of this may be an antibody-bound cellulose paper contained within a hollow channel of the punctal insert.

Using an antibody may allow for specific capture of 1 targeted protein of the approximately 1500 proteins that may be present in tears. The targeted proteins may be free proteins or proteins bound to the surface of a biological particle. For example, the biological particle may be an extracellular vesicle, a virus, a bacterium, or the like. Exosomes are a type of extracellular vesicle of particular interest because the exosomes of breast cancer patients have been found to contain specific breast cancer biomarkers

In another example, to determine if someone has COVID-19 antibodies in their tears, the spike protein of SARS-COV-2 may be incorporated into the punctal insert. The punctal insert may then scavenge any SARS-COV-2 antibodies that flow through it.

In another example, to capture a specific strand of DNA present in tears, the complementary DNA strand may be used in the punctal insert.

The capture agent may include a metal-binding ligand, an aptamer, or DNAzymes. The aptamer may be a polypeptide or polynucleotide. One way of synthesizing an aptamer or DNAzyme may be through the SELEX process (as well known in the art).

Importantly, in all cases, the capture agent is immobilized on the surface of a scaffold material to ensure the capture agent remains contained within or on the punctal insert in the presence of continuously draining tear fluid. In some embodiments the scaffold material may be embedded within an open channel that penetrates through the punctal insert. In other embodiments, the punctal insert itself may act as the scaffold material. For example, the punctal insert may be entirely made out of a microporous hydrogel or foam. Typical materials that may be used as the scaffold are silicones, silica, metal oxides, metal phosphates and biocompatible polymers such as poly(HEMA), polysaccharide based polymers (e.g. cellulose, agar, alginic acid, and chitosan), polypeptide based polymers or gels (e.g. gelatin or crosslinked proteins such as bovine serum albumin).

The scaffold material of the punctal insert may consist of labeled microparticles. For example, labeled microparticles may be fluorescently or colorimetrically tagged microspheres. An example of commercially available fluorescently-tagged beads may be Luminex MagPlex Microspheres. Labeled microparticles are particularly convenient for analysis as they can be easily purified via centrifugation, or magnetic separation in the case of magnetic microparticles, as well as aliquoted for multiple analyses from the same sample. To prevent microparticles from leaching out of the punctal insert during in vivo analyte capture, the labeled microparticles may be contained within the open channels of the insert by one or more membranes, which may be removed after removing the punctal insert from the patient to release the microparticles for in vitro analysis.

In addition to fluorescent or colorimetric dyes, microparticles may be labeled by a compound that is released and recognized by a chromatograph and/or mass spectrometer, an enzyme, fluorescent quantum dots, or a chemo-, electro- or photo-luminescent particle or dye.

The capture-agent-immobilized scaffold material may contain a surface-bound blocking agent that prevents non-specific binding of proteins in tears. This may also be known as biofouling. Typical fouling resistant blocking agents may be bovine serum albumin (BSA), polyethylene glycol, zwitterionic polymers, and the like.

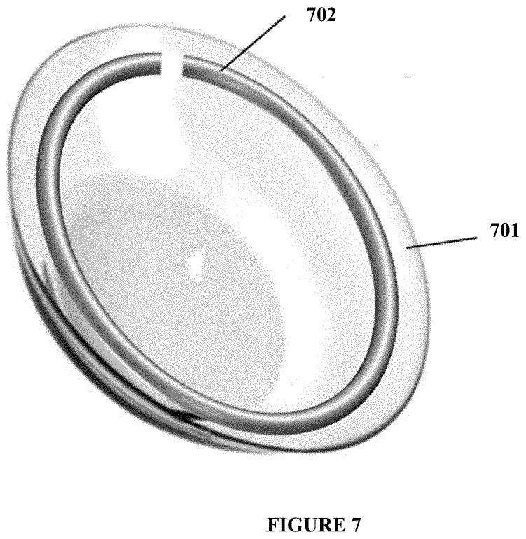

FIG. 7 is an example of a contact lens used for analyte capture. The contact lens 701 may include an open channel 702. The open channel 702 may contain one or more surface-immobilized capture agents.

In an example method for in vivo capture of an analyte and subsequent in vitro analysis of the analyte, the punctal insert may be inserted into one or more lacrimal puncta of a host enabling collection of target analyte(s) for a predetermined period of time (for example, 15 minutes for a quick analysis to days or weeks for a time-averaged analysis of a patient's tear biomarker profile). The punctal insert(s) may then be removed and the contents of the punctal insert(s) analyzed. The analysis method depends on the nature of the one or more capture agent(s). In cases where the capture agents consist of antibodies, the captured biomarkers may be directly analyzed via a colorimetric, fluorescence, electroluminescence, and photo-luminescence or chemoluminescence immunoassay. Alternatively, captured biomarkers may first be released from antibodies via an eluting solution (for example, 6M guanidine-HCl) and subsequently analyzed via liquid chromatography mass spectrometry (LC-MS), MALDI-TOF or gel electrophoresis.

A visible cue of a health condition, for example, a change in color or fluorescence, may be generated, after which the punctal plug may be removed and the contents of the punctal insert(s) analyzed.

The punctal inserts may be analyzed using polymerase chain reaction (PCR), reverse transcription PCR (RT-PCR), nicking enzyme amplification reaction (NEAR), loop mediated isothermal amplification (LAMP), RT-LAMP, enzyme-linked immunosorbant assay (ELISA), or genomic sequencing.

The punctal inserts may be analyzed using nuclear magnetic resonance (NMR), mass spectrometry, high performance liquid chromatography (HPLC), and/or elemental analysis.

In an example method of analyte detection and/or quantification in a bodily fluid (for example, tears, saliva, blood, sweat or urine), a sensor, specifically containing DNAzymes, may be put in contact with bodily fluid, removed, and the contents analyzed. The content may be analyzed to determine if either any of the DNAzymes reacted with an analyte (qualitatively) and/or the concentration or number of DNAzymes that reacted with an analyte (quantitatively), while the DNAzyme-sensor was in contact with the bodily fluid.

The sensor may be put into contact with a bodily fluid so as to undergo reaction in vivo in the presence of a specific analyte (if that analyte is present), and subsequently, the DNAzyme device may be removed for in vitro analysis.

In another example method of analyte detection, cleavage of a certain concentration of DNAzymes and/or aptamers in a DNAzyme-linked and/or aptamer-linked hydrogel sensor leads to a colorimetric signal may be detectable by the naked eye. After the colorimetric signal is detected, the contents of the sensor may be analyzed as previously described herein.

The material comprised of DNAzymes and/or aptamers may be analyzed using NMR, mass spectrometry, HPLC, PCR, RT-PCR, NEAR, LAMP, RT-LAMP, DNA sequencing, RNA sequencing, and/or elemental analysis.

In another example, a colorimetric device for optical detection of an analyte may be described herein. The colorimetric device may be comprised of a fluorescent or luminescent material or a material of an easily distinguishable pattern or color. Overlaying this optically distinctive material may be a hydrogel that conceals the visually distinctive material from a detector and/or the naked eye. The material may be crosslinked by DNAzymes and/or aptamers that cleave upon interaction with the analyte. Upon cleavage of a significant amount of DNAzyme (and/or aptamer) crosslinks, the concealing hydrogel may wash away, dissolve or be collected in a separate chamber of the device, thereby revealing the optically distinctive material to the optical detector and/or naked eye.

The colorimetric device may be embedded within a permeable lacrimal insert. The colorimetric device may be designed to display an optical signal in response to an analyte in tear fluid.

In an example method, once the ocular insert is removed from the patient, all loosely bound tear constituents may be washed out of the insert to yield a purified sample of concentrated targeted biomarkers ready for analysis. Specifically for protein capture, performing an immunoassay directly on the capture-agent immobilized scaffold material. An example of a sandwich-type immunoassay adapted for our system is may be as described herein.

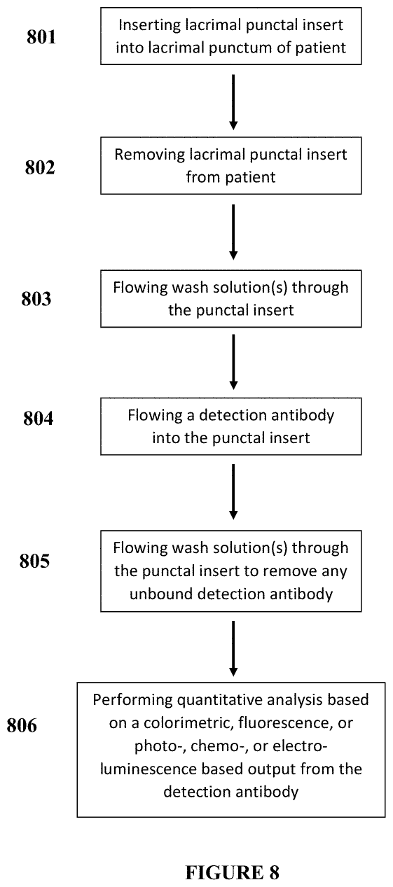

FIG. 8 is an example method of analyte capture using a lacrimal punctum insert. A lacrimal punctal insert may be inserted into the lacrimal punctum of a patient 801. The lacrimal punctum insert may then be removed from the patient 802. A washing solution may flow through the lacrimal punctum insert 803. A detection antibody may the flow into the lacrimal punctum insert 804. Another wash solution may flow through the lacrimal punctum insert to remove any unbound detection antibody 805. Finally a quantitative analysis may be performed based on a colorimetric, fluorescence, or photo-, chemo-, or electro-luminescence based output from the detection antibody 806.

For example, the lacrimal punctal insert may contain microparticles as the capture agent scaffold. After removing the lacrimal punctal insert from the patient, one end of the insert is either punctured or a membrane at one end is physically removed to release the particles for performing flow cytometry and/or a bioassay.

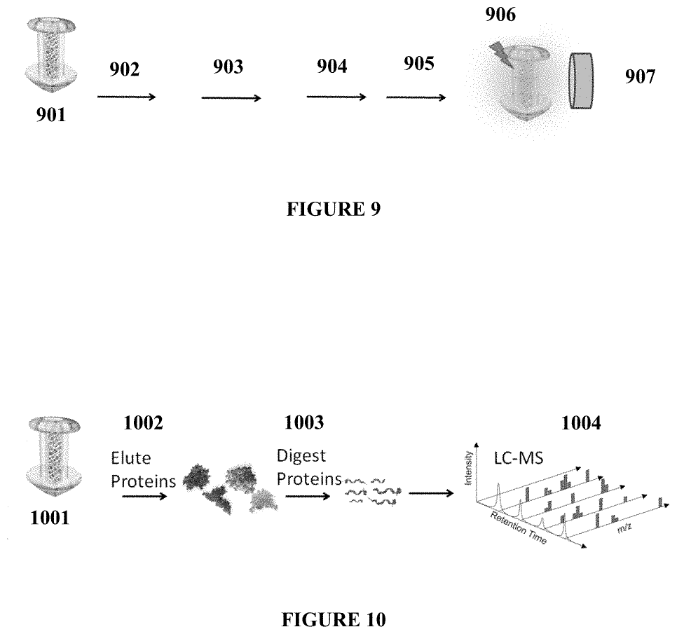

FIG. 9 is an example illustration of what this may look like using a fluorescence-based immunoassay. The lacrimal punctal insert may be removed from a patient 901. A washing solution may flow through the lacrimal punctum insert 902. A fluorophore-tagged detection antibody may be introduced in the lacrimal punctal insert 903. Another wash solution may flow through the lacrimal punctum insert to remove any unbound detection antibody 904. The fluorescence may then be analyzed 905. Finally, a light source (not shown) may be used to excite the antibody-bound fluorophore, and a detector 907 may be used for fluorescence analysis 906.

In some embodiments the detection antibody may already be tagged with the fluorescence, color or luminescence inducing moiety. Alternatively, the detection antibody may be labeled with a reactive functional group, in which case additional steps may be added for introducing a fluorescence/colorimetric/luminescence generating moiety that then binds with the functional group on the detection antibody (these variations are all well known in the art). For example, using a biotin-labeled detection antibody and a streptavidin labeled dye.

In another example, specific to aptamers or DNAzymes, attaching a FRET-based dye system (as is well known in the art) that produces a fluorescence signal upon binding of the target analyte may be used. In this example, a signal may be produced in vivo upon binding of the analyte with the capture agent. The signal may be observed both by eye qualitatively as well as quantitatively via in vitro analysis.

In another method, a bioassay may be performed directly on the punctal insert. This method may be particularly useful for inserts that are transparent (for example, silicone) but may also be done on light-scattering inserts. In one example of this method, the lacrimal punctal insert may be removed from the patient and inserted into a microfluidic device. The microfluidic device may be used to inject solutions to perform a subsequent bioassay. For example, the microfluidic device may be used to efficiently wash out any unbound tear constituents as well as flow in reagents for performing the bioassay. In specific cases where the captured analytes are meant to be removed prior to analysis (for example, for performing LC-MS) the microfluidic device could also inject an eluting buffer to release the captured analytes from the punctal insert.

In some embodiments, a microfluidic device may also contain the components necessary to perform the bioassay immediately after the reagents are introduced. For example, a microfluidic device with an integrated light source and photodetector could be used to perform a fluorescence immunoassay.

FIG. 10 is an example illustration of releasing analytes from a capture agent. The analytes, proteins in this example are released 1002 from the lacrimal punctal insert 1001. The released proteins 1002 may be digested 1003 and then analyzed via liquid chromatography mass spectrometry (LC-MS) 1004. Many other analysis methods including gel electrophoresis, other mass spectrometry methods (such as MALDI-TOF), and other methods common for protein detection may be used.

In another example method, the scaffold material may first be removed or exposed from the insert. A bioassay may then be performed on the removed/exposed scaffold.

The capture agent of the lacrimal punctal insert may be bound to a surface on or within the punctal via a cleavable bond. A cleavable bond may be any bond that can be cleaved under relatively mild conditions. For example, the capture agent may be bound to a surface via a disulfide bond, which is relatively stable in vivo. After removing the lacrimal punctal insert from the patient, the disulfide bond may be cleaved under reducing conditions (for example, using dithioreitol or TCEP) to release the capture agents along with any analytes bound to the capture agents.

In some embodiments, two or more types of capture agents may be bound to the ocular insert in order to simultaneously capture two more specific analytes in tear fluid. After removing the insert from the patient, the concentrations of the two or more specific analytes may be quantitated with respect to one another in order to yield a multiplex bioassay.

What is claimed:

1. An lacrimal punctal insert for capturing and concentrating one or more target analytes in vivo in bodily fluid comprising:
one or more surface-bound capture agents, wherein each of the one or more surface-bound capture agents specifically binds and concentrates at least one target analyte from tear fluid.

2. The lacrimal punctal insert of claim 1 wherein the surface-bound capture agent is comprised of at least one of an antibody that binds one or more specific proteins, a protein that binds one or more specific proteins, ions, oligonucleotides and/or polynucleotides, an oligonucleotide or polynucleotide that binds one or more specific oligonucleotides or polynucleotides, or a molecularly imprinted polymer.

3. The lacrimal punctal insert of claim 1 wherein the capture agent is comprised of at least one of a metal-binding ligand, an aptamer, a DNAzyme or, molecularly imprinted polymers.

4. The lacrimal punctal insert of claim 1 further comprising:
one or more hollow channels, wherein the one or more hollow channels contain the surface-bound capture agent, enabling the tear fluid to access capture agents on interior surfaces of the lacrimal punctal insert.

5. The lacrimal punctal insert of claim 1, further comprising:
a porous material wherein pores contain the surface-bound capture agent, enabling the tear fluid to access capture agents on interior surfaces of the lacrimal punctal insert.

6. The lacrimal punctal insert of claim 4, wherein the one or more hollow channel extend continuously through the punctal insert, enabling the tear fluid to drain naturally through the lacrimal punctal insert into a lacrimal canaliculi.

7. The lacrimal punctal insert of claim 4, wherein the hollow channel do not fully permeate the lacrimal punctal insert, so the lacrimal punctal insert may act as a punctal plug to block the drainage of tear fluid through the lacrimal canaliculi.

8. The lacrimal punctal insert of claim 1, wherein the at least one target analyte is at least one of an organic compound, a biomarker, pharmacological agent, a synthetic organic compound, an environmental pathogen, a metal ion, a virus, a bacteria, a fungus, an enzyme, a metabolite, a lipid, a phospholipid, a glycolipid, an extracellular vesicle, an oligonucleotide, a polynucleotide, microRNA, a protein, and a peptide.

9. The lacrimal punctal insert of claim 4, wherein the capture agent is bound to one or more separate scaffold materials embedded within the channels and pores of the lacrimal punctal insert.

10. The lacrimal punctal insert of claim 9, wherein at least one of the scaffold materials is comprised of microparticle or labeled microparticles.

11. The lacrimal punctal insert of claim 10, wherein the labeled microparticles are contained within the open channels by a removable membrane, which upon removing, enables the microparticles to be released for in vitro analysis.

12. The lacrimal punctal insert of claim 1 wherein the surface-bound capture agent is a small molecule, a drug, a metabolite, an anion, a cation, a charged polymer, an oligosaccharide, a polysaccharide, a lipid, a glycolipid, a phospholipid, a metallic surface, a metal oxide or a combination thereof.

13. The lacrimal punctal insert of claim 1 wherein the capture agent is comprised viral capture nanoparticles or microparticles.

14. A method for analyte capture using an ocular insert, the method comprising:
inserting an ocular insert into a portion of an eye of a patient, wherein the ocular insert is comprised of one or more surface-bound capture agents, wherein the surface-bound capture agents binds to at least one target analyte from tear fluid;
removing the ocular insert from the patient; and
performing an assay to analyze the composition of the ocular insert.

15. The method of claim 14, wherein the ocular insert is a lacrimal punctal insert for insertion into a lacrimal punctum and contains one or more surface-bound capture agents.

16. The method of claim 14, wherein the ocular insert is a contact lens containing one more surface-bound capture agents.

17. The method of claim 14 wherein the ocular insert fits into a conjunctival sac of the patient and contains one or more surface-bound capture agents.

18. The method of claim 14 further comprising:
removing the ocular insert from the patient;
washing the ocular insert to remove any non-specifically bound tear constituents; and
performing a bioassay to analyze the remaining analytes specifically bound to the one or more capture agents.

19. The method of claim 14 further comprising:
extracting the one or more analytes specifically-bound to the one or more capture from the ocular insert via an eluting buffer; and
analyzing the one or more analytes.

20. The method of claim 19 where the extracted analytes are analyzed via liquid chromatography, mass spectrometry, gel electrophoresis, or a combination thereof.

21. The method of claim 14, wherein a colorimetric, fluorescence, or chemo-, electro-, or photo-luminescence bioassay is performed to analyze the constituents collected by the ocular insert.

22. The method of claim 14, wherein one or more capture agents are designed to capture one or more oligonucleotides or polynucleotides, wherein PCR, RT-PCR, LAMP, RT-LAMP or gene sequencing are performed to analyze the constituents collected by the ocular insert.

23. The method of claim 14, wherein one or more of the capture agents are comprised of an oligonucleotide or polynucleotide, wherein the one or more oligonucleotide or polynucleotide capture agents are released from the ocular insert and analyzed by PCR, RT-PCR, LAMP, RT-LAMP or gene sequencing.

24. The method of claim 14, further comprising:
removing the ocular insert from the patient;
inserting the ocular insert into a microfluidic device; and
injecting, using the microfluidic device, one or more washing buffers, reagents, eluting buffers or a combination thereof, into the ocular insert, prior to performing a bioassay.

25. The method of claim 14, wherein the one or more surface-bound capture agents are removed from the ocular insert prior to performing a bioassay.

26. The method of claim 23, wherein the one or more capture agents are bound to the surface of the ocular insert and/or a porous scaffold material within the ocular insert via a cleavable bond, wherein the cleavable bond is cleaved to release the surface-bound capture agents to perform a bioassay.

27. The method of claim 23, wherein the ocular insert comprises microparticles as a capture-agent-immobilizing scaffold, wherein prior to performing a bioassay.

28. The method of claim 27 further comprising:
releasing the microparticles from the ocular insert, either by puncturing the ocular insert or removing a removable membrane designed to contain the microparticles within the ocular insert; and
performing flow cytometry, a fluorescence, colorimetric, chemo-, electro-, or photo-luminescence assay, or a combination thereof, on the released microparticles.

29. The method of claim 14 wherein at least two distinct analytes are specifically captured by the ocular insert, wherein the ocular insert is first removed from the patient, and the concentrations of the two or more distinct analytes are measured with respect to one another in order to yield a multiplex bioassay.
